(19) United States
(12) Patent Application Publication
(10) Pub. No.: US 2008/0231804 A1
(43) Pub. Date: Sep. 25, 2008

(54) Vivo Spatial Measurement of the Density and Proportions of Human Visual Pigments

(76) Inventors: Simon Gagne, Saint-Antoine-de-Tilly (CA); Sylvain Comtois, Quebec (CA)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 12/092,268

(22) PCT Filed: Nov. 8, 2006
(86) PCT No.: PCT/CA06/01831

§ 371 Date: Apr. 30, 2008

Related U.S. Application Data
(60) Provisional application No. 60/734,319, filed on Nov. 8, 2005.

(51) Int. Cl. A61B 3/14; A61B 3/15; A61B 3/12

(52) U.S. Cl. 351/208; 351/246; 351/206

(57) ABSTRACT

The present invention concerns a method and system for in vivo spatial measurement of density and relative proportions of retinal visual pigments. The method involves the steps of illuminating a retina with light of a given intensity and wavelength, acquiring the residual light coming from the retina using a photosensing device having an array of pixels, attributing a residual intensity to each pixel thereby producing a corresponding spatial image of the retina, and posing an equation relating the residual intensity to a number of unknown variables of interest. The above steps are repeated using light of a different wavelength but same intensity to acquire a set of spatial images and a set of corresponding equations for each pixel of each image. For each pixel of each image, the set of equations is solved for the unknown variables obtaining the spatial measurement of density and relative proportions of retinal visual pigments.

### FIELD OF THE INVENTION

The present invention relates to a system and method for in vivo spatial measurement of density and proportions of human retinal visual pigments.

### BACKGROUND OF THE INVENTION

The back of the human eye is lined with two groups of photoreceptors: cones and rods. These cells capture the light from the world around us and give rise to colour vision under high brightness (day vision: cones) and to black and white vision under low brightness (night vision: rods). The distribution of the photoreceptors (density) varies spatially. The region of clear image vision, the central region, formed of the macula and the fovea is mainly made up of cones whereas the peripheral region is mainly made up of rods. It has been possible to determine the proportion within the eye of each type of photoreceptor using histological methods (R. W. Rodieck, The First Steps in Seeing, Sinauer Associates Inc., 562 pages, 1998). However, it is only recently that a method has been developed for measuring in vivo the arrangement of the three types of cones in the retina—thanks to an ophthalmoscope developed by David Williams of Rochester that resolves the photoreceptors using adaptive optics (A. Roorda, A. B. Metha, P. Lennie, and D. R. Williams, “Packing arrangement of the three cone classes in primate retina”, Vision Res. 41, 1291-1306, 2001). Despite the incredible precision of this method, the density of the visual pigment of each photoreceptor cannot be measured.

Many devices have been developed for measuring the density of visual pigments in the eye (C. Hood, and W. A. H Rushton, “The Florida retinal densitometer”, J. Physiol. 217, 213-219,1971; D. van Norren and J. A. van der Kraats, “Continuously recording retinal densitometer”, Vision Res. 21, 897-905, 1981; U. B. Sheorey, “Clinical assessment of rhodopsin in eye”, Brit. J Ophtalmol. 60, 135-141, 1976; I. Fram, J. S. Read, B. H. McCormick, and G. A. Fishman, “In vivo study of the photolabile visual pigment utilizing the television ophthalmoscope image processor”, Computers in Ophtalmol. Avril, 133-144, 1979; P. E. Kilbride, M. Fishman, G. A. Fishman, and L. P. Hutman, “Foveal cone pigment density difference in the aging human eye”, Vision Res. 26, 321-325, 1983; D. J. Faulkner, C. M. Kemp, “Human rhodopsin measurement using a TV-based imaging fundus reflectometer”, Vision Res. 24, 221-231, 1984; D. van Norren and J. van der Kraats, “Imaging retinal densitometry with a confocal scanning laser ophthalmoscope”, Vision Res. 29, 369-374, 1989; J. Fortin, Évaluation non effractive des pigments visuels au moyen d'un densimètre à images video, PhD Thesis, Laval University (Canada), 1992; J. van de Kraats, T. T. J. M. Berendschot, and D. van Norren, “The pathways of light measured in fundus reflectometry” Vision Res. 36, 2229-2249, 1996). They all operate on the same principle, which is illustrated in FIG. 1, sending a light into the eye (L) and analysing the light that comes back out (R).

The light directed to the eye (L) can contain several components of varying intensity (Iᵢ) that are each a function of time (t) and wavelength (λ). We can therefore write:

$$L = {\sum }_{i} {I}_{i} ( λ , t )$$

However, the light exiting the eye is of a more complex nature since it depends on the multiple reflections and absorptions that are produced in the different media found in the interior of the eye. FIG. 1 shows the pertinent media:

Visual pigments: pigments found in the photoreceptors (cones and rods) that give rise to the vision process once they absorb the light. It is the density of these pigments that the densitometer is expected to measure.

Pigment epithelium: layer of cells containing a pigment that absorbs almost all of the light that is not captured by the visual pigments found in the photoreceptors. These cells have an important role in the regeneration of visual pigment and allow the increase of the spatial contrast of images.

Ocular medium: consists of all of the structures other than those already mentioned: the vitreous humour, the aqueous humour, the lens, all of the surfaces having media with different indices of refraction, the cornea, etc.

The light coming out of the eye at a given wavelength (Iᵣ(λ)) for a given incident light (Iᵢₙ(λ)) is:

Iᵣ(λ)=[Tₘₒ²(λ)Tₚᵥ²(λ)Rₑₚ(λ)+R]Iᵢₙ(λ) Equation (1)

where: Tₘₒ²=transmission of ocular media
Tₚᵥ²=transmission of visual pigment
Rₑₚ=reflection of the pigment epithelium
R=term combining the diffuse light and the non-Lambertian reflection in the ocular medium (independent of the wavelength)

It is worth noting that the transmission terms are squared owing to the light which crosses the relevant structures twice. The term of interest here is that of the transmission of the visual pigment (Tₚᵥ²). Several unknowns in Equation (1) can be regrouped such that the light exiting the eye is expressed as follows:

$$\frac{{I}_{r} ( λ )}{{I}_{i n} ( λ )} = A ( λ ) {{T}_{pv} ( λ )}^{2} + K \qquad Equation ( 2 )$$

where A(λ)=Tₘₒ(λ)²Rₑₚ(λ) and K=R, this term is called parasitic light.

Equation (2) therefore contains three unknowns. Presently, there is no known method for taking three measurements thus solving this equation. The usual procedure consists firstly of bleaching the visual pigment with the help of a bright light and of taking two measurements in sequence: the first right after the bleaching and the other after the visual pigments have regenerated (≈20 minutes). It is worth noting that the light incident on the eye (Iᵢₙ(λ)) must be the same during the two measurements. During the first measurement, due to bleaching, the visual pigment is transparent (Tₚᵥ²=1). We therefore have the following equations:

$$\begin{matrix}\frac{{I}_{r} ( {λ}_{i} )}{{I}_{i n} ( {λ}_{i} )} = A ( {λ}_{i} ) K & Equation ( 3 ) \\ \frac{{I}_{r} ( {λ}_{i} )}{{I}_{i n} ( {λ}_{i} )} = A ( {λ}_{i} ) {T}_{pv}^{2} + K \\ Solving for {T}_{pv}^{2} \text{:} & Equation ( 4 ) \\ {T}_{pv}^{2} = \frac{\frac{{I}_{r} ( {λ}_{j} )}{{I}_{i n} ( {λ}_{j} )} - K}{\frac{{I}_{r} ( {λ}_{i} )}{{I}_{i n} ( {λ}_{i} )} - K} & Equation ( 5 )\end{matrix}$$

Equation (5) will always give a value of Tₚᵥ² less than that required, regardless of the value of K, since the denominator is greater than the numerator. Of course, the measured value is only exact when the term of the parasitic light (K) is zero and A(λᵢ) is not wavelength dependent. Nevertheless, it should be noted that the method measures only the average transmission of the visual pigments. When the measurement region contains both cones and rods, the measurement depends on their respective proportions. Generally, researchers in the domain measure regions containing mainly cones (fovea) or regions rich in rods (periphery). The solution of Equation (5) is given here in terms of transmission of pigments (Tₚᵥ) rather than in terms of density. Generally, the term density (D) is used when taking these measurements (whence the terms densitometer, densimeter, densitometry, and densimetry). The use of the term “density” rather than the term “transmission” comes from a mathematical convenience and does not change in any way the mathematical analysis carried out here. The reason being that density is a logarithmic value and can therefore be added, as in the case for successive optical media and unlike the case of transmission values which must be multiplied. The density is defined as being:

D=log₁₀(1/T)

where

$$D = \frac{1}{2} {log}_{10} \frac{[ \frac{{I}_{r} ( {λ}_{i} )}{{I}_{i n} ( {λ}_{i} )} - K ]}{[ \frac{{I}_{r} ( {λ}_{j} )}{{I}_{i n} ( {λ}_{j} )} - K ]} \qquad Equation ( 6 )$$

Many instruments, as described above, have been developed for measuring in vivo either the density of cones or the density of rods. However, there does not exist any method permitting to measure spatially in vivo the density and the proportion of the cones and rods. The method and system described herein permits such measurements.

### SUMMARY OF THE INVENTION

It is an object of the present invention to propose a method and system for obtaining an in-vivo spatial measurement of a retina of an eye of a patient representative of density and relative proportions of visual pigments in the retina.

In accordance with one aspect of the present invention, there is therefore provided a method for obtaining an in-vivo spatial measurement of a retina of an eye of a patient representative of density and relative proportions of visual pigments in the retina. The method includes the steps of:
(a) illuminating the retina with a light beam of a given incident intensity Iᵢₙ(λᵢ) and a given wavelength λᵢ;
(b) detecting a residual light beam coming from the retina and acquiring light data from the residual light beam using a photosensing device having a bidimensionnal array of pixels;
(c) processing the light data acquired by the photosensing device to attribute a residual intensity Iᵣ(λᵢ) of the residual light beam to each of the pixels, thereby producing a corresponding spatial image of the retina;
(d) for each pixel, posing an equation relating the residual intensity Iᵣ(λᵢ) to a number N of unknown variables of interest representative of the density and relative proportions of the visual pigments;
(e) repeating steps (a) through (d) for a number N of image acquisitions, the illuminating the retina including projecting a light beam of a different wavelength λᵢ and a same incident intensity Iᵢₙ(λᵢ) onto the retina for each acquisition; and
(f) for each pixel, numerically solving a set of N equations obtained through step (e) for the unknown variables to obtain therefrom the in-vivo spatial measurement of the retina representative of the density and relative proportions of the visual pigments in the retina.

According to one embodiment of the method, the equation posed in step (d) relating the residual intensity Iᵣ(λᵢ) to the density and relative proportions of the visual pigments is:

$$\frac{{I}_{r} ( {λ}_{i} )}{{I}_{i n} ( {λ}_{i} )} = F ( {λ}_{i} ) A [ {a ( {TP}^{ n} )}^{2} + ( 1 - a ) {( {TS}^{ m} )}^{2} ] + K$$

where F(λᵢ) represents a normalized reflection for a wavelength λᵢ with respect to a wavelength λⱼ following bleaching of the visual pigments, A is an absorption factor, a accounts for relative proportion of cones with respect to rods, TP accounts for cone sensitivity, TS accounts for rod sensitivity, n and m are exponents measured respectively from sensitivity curves for scotopic and photopic vision at the given wavelength λ_{b} and K accounts for a contribution from parasitic light. Preferably, values for F(λᵢ) are determined from a known normalized reflection curve. The number N of unknown variables may be five and the unknown variables may be A, a, K, TS, and TP.

According to another embodiment of the method, the equation posed in step (d) relating the residual intensity Iᵣ(λᵢ) to the density and relative proportions of the visual pigments is:

$$\frac{{I}_{r} ( {λ}_{i} )}{{I}_{i n} ( {λ}_{i} )} = ( \frac{{I}_{rbleached} ( {λ}_{i} )}{{I}_{i n} ( {λ}_{i} )} - K ) [ {a ( {TP}^{ n} )}^{2} + ( 1 - a ) {( {TS}^{ m} )}^{2} ] + K$$

where I_{rbleached}(λᵢ) is the residual intensity of the residual light beam coming from the retina when in a bleached state, a accounts for relative proportion of cones with respect to rods, TP accounts for cone sensitivity, TS accounts for rod sensitivity, n and m are exponents measured respectively from sensitivity curves for scotopic and photopic vision at the given wavelength λᵢ, and K accounts for a contribution from parasitic light.

According to the latter embodiment of the method, the method may further include an additional step before step (f) of determining I_{rbleached}(λᵢ) through observation of the retina in a bleached state. Preferably, the additional step includes the substeps of:
(i) bleaching the retina;
(ii) illuminating the bleached retina with a light beam of a given incident intensity Iᵢₙ(λᵢ) and a given wavelength λᵢ;
(iii) detecting a residual light beam coming from said bleached retina and acquiring light data from said residual light beam using a photosensing device having a bidimensionnal array of pixels;
(iv) processing said light data acquired by said photosensing device to attribute a residual intensity I_{rbleached}(λᵢ) of said residual light beam to each of said pixels thereby producing a corresponding spatial image of said retina;
(v) repeating steps (i) through (v) for a number N of image acquisitions, said illuminating said retina comprising projecting a light beam of a different wavelength λᵢ and a same incident intensity Iᵢₙ(λᵢ) onto said retina for each acquisition, wherein said different wavelengths λᵢ each corresponds to one of the different wavelengths λᵢ of step (e).

The number N of unknown variables may be four and the unknown variables may be a, K, TS, and TP.

According to another aspect of the invention, there is provided a system for in vivo spatial measurement of a retina of an eye of a patient representative of density and relative proportions of visual pigments in the retina. The system includes: illumination means for illuminating the retina with light of a given intensity Iᵢₙ(λ) and a given wavelength λ; a light data acquisition system including a photosensing device for detecting a residual light beam coming from the retina and acquiring corresponding light data, the photosensing device having a bidimensionnal array of pixels, a processor for processing light data acquired by each pixel of the photosensing device and attributing a residual intensity Iᵣ(λ) of the residual light beam to each of the pixels thereby producing a corresponding spatial image of the retina, and a controller for controllably producing a number N of spatial images of the retina, each spatial image produced using the illumination means with light of a different given wavelength and same given incident intensity for each image; and a data analyser for numerically analysing each pixel of each of the number N of spatial images of the retina, the data analyser posing an equation for each pixel relating the residual intensity Iᵣ(λ) to a number N of unknown variables of interest representative of the density and relative proportions of the visual pigments and numerically solving for each pixel a set of N equations for the unknown variables to obtain therefrom the in-vivo spatial measurement of the retina representative of the density and relative proportions of the visual pigments in the retina.

According to one embodiment of the system, the illumination means includes a light source. Preferably, the light source includes a source of visible light. Advantageously, the illumination means may include at least one interferential filter for selecting the light of a given wavelength. The data analyser may preferably include computer means.

According to another embodiment of the system, the system may include an ophthalmoscopic camera which incorporates said illumination means. In addition, the system may include a charge-coupled device (CCD) fundus camera which incorporates the photosensing device and the processor. Furthermore, the system may include image alignment means for controllably aligning the ophthalmoscopic camera with the eye.

### DESCRIPTION OF THE FIGURES

Further aspects and advantages of the invention will be better understood upon reading the description of preferred embodiments thereof with reference to the following drawings:

FIG. 1 is a schematic diagram of the eye showing the multiple reflections and transmissions of light that are produced by the different media found in the interior of the eye. [Prior Art]

FIG. 2 is a graph of photoreceptor sensitivity versus wavelength: the curve on the left is associated with the rods (scotopic or night vision) and that on the right is associated with the cones (photopic or day vision). [Prior Art]

FIG. 3 is a graph of the reflection intensity from the back of the eye versus wavelength following bleaching of the visual pigment. [Prior Art]

FIG. 4 is a thee-dimensional graph showing how the density solution of cones (TP) and rods (TS) are computed. Such a computation is done for each point in the retina.

FIG. 5 is an example of a series of six CCD camera images obtained according to one embodiment of the invention, showing the residual intensity profile information of line 150 of each of five images of a retina. The five images of the retina are obtained using light beams of a same intensity and following incident wavelengths: 470 nm, 500 nm, 530 nm, 560 nm and 600 nm. The sixth image (taken with the CCD camera in darkness) shows noise generated by the CCD camera, which is used to correct for noise in the images of the retina.

FIG. 6 is an example of spatial measurements of the retina representative of density (TS, TP) and relative proportions of visual pigments in the retina (a) as well as spatial measurements representative of the characteristics of the back of the eye (A) and parasitic light (K), obtained from the images of FIG. 5. The values of TS, TP, a, A, and K for the pixels of line 120 are shown graphically.

FIG. 7 is a schematic diagram of an eye of a human subject showing the three reflections used in image alignment.

FIG. 8A is a schematic side view diagram of the invention according to one aspect of the invention, showing illumination means and a light data acquisition system.

FIG. 8B is a front view of an alignment means shown in FIG. 8A.

### DESCRIPTION OF A PREFERRED EMBODIMENT OF THE INVENTION

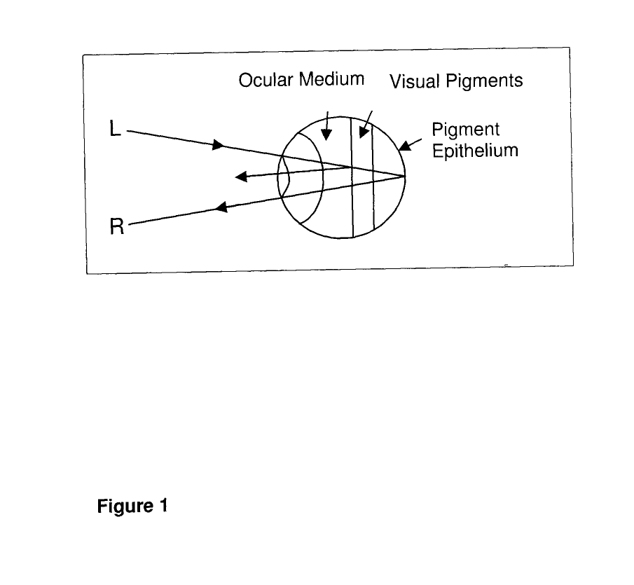

The aspects of the present invention will be described more fully hereinafter with reference to the accompanying drawings, FIGS. 1 to 8, in which like numerals refer to like elements throughout. The terms images, pictures, photos, and photographs are used interchangeably herein to denote a representative reproduction of an object, and includes images obtained by digital means.

### General Description

In accordance with one aspect of the present invention, there is generally provided a method for obtaining an in vivo spatial measurement of a retina of an eye of a patient representative of the density and relative proportions of visual pigments in the retina, which includes the following steps.
(a) Illuminating said retina with a light beam of a given incident intensity Iᵢₙ(λᵢ) and a given wavelength λᵢ

To illuminate the retina, a light source may be used to project a light beam of a given incident intensity and given wavelength through a pupil of the eye onto the retina. The light source used preferably includes a source of visible light. The source of visible light may be a source of monochromatic visible light, as in the case of a laser. It is to be understood that the term “monochromatic visible light” refers to visible light of a single colour, that is to say, radiation in the visible electromagnetic spectrum of a single wavelength as well as radiation in the visible electromagnetic spectrum of a narrow wavelength band so as to be considered a single wavelength in practice. Alternatively, the source may be a source of polychromatic visible light, as in the case of a light source of white light. Here, it is to be understood that the term “polychromatic visible light” refers to visible light of many colours, that is to say, radiation in the visible electromagnetic spectrum of more than one wavelength, in practice.

Interferential filters may be used to select a light of a given wavelength λᵢ.

A calibration photometer may be used to select the incident intensity Iᵢₙ(λᵢ) of the light.

Advantageously, the illumination may be accomplished using the light source found in an ophthalmoscopic camera used to view the eye of the patient.
(b) Detecting a residual light beam coming from the retina and acquiring light data from the residual light beam using a photosensing device having a bidimensionnal array of pixels

The detecting of a residual light beam coming from the retina and acquiring light data from this residual light beam may be done using a charge-coupled device (CCD) as the photosensing device. A charge-coupled device (CCD) typically consists of an integrated circuit containing an array of linked, or coupled, light sensitive pixels which sense light through the photoelectric effect. The integrated circuit records the intensity of light as a variable electric charge. Their charges may then be equated to shades of light for monochrome images or shades of red, green and blue when used with color filters.
(c) Processing the light data acquired by the photosensing device to attribute a residual intensity Iᵣ(λᵢ) of the residual light beam to each of the pixels, thereby producing a corresponding spatial image of the retina

The processing of the light data acquired from the photosensing device may be carried out using an analog-to-digital converter to transform the charges into binary data. The binary data may then be processed by electronic circuitry found in a computer.

Of course, a CCD fundus camera may be used to accomplish both the detecting of step (b) and the processing of step (c).

The term “pixel” is used herein to refer interchangeably to both the smallest detection elements of the photosensing device as well as the smallest resolved elements of the image produced by the photosensing device.
(d) For each pixel, posing an equation relating the residual intensity Iᵣ(λᵢ) to a number N of unknown variables of interest representative of the density and relative proportions of the visual pigments

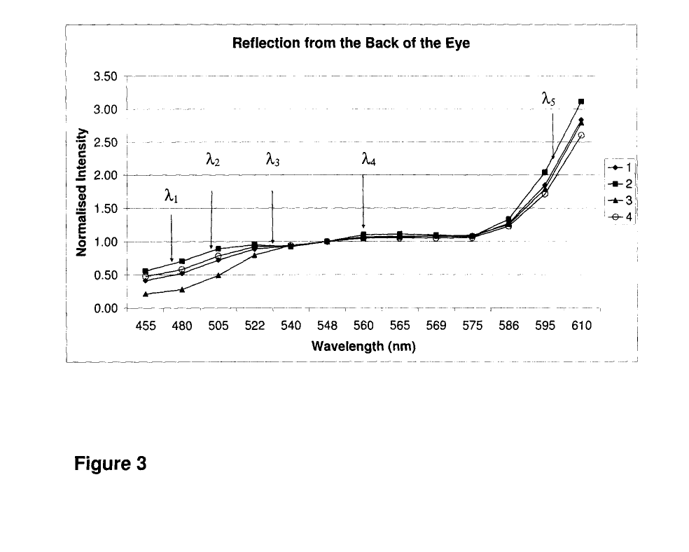

When bleaching of the retina of the patient is not feasibly possible, the equation posed in step (d) relating the residual intensity Iᵣ(λᵢ) to the density and relative proportions of the visual pigments is:

$$\frac{{I}_{r} ( {λ}_{i} )}{{I}_{i n} ( {λ}_{i} )} = F ( {λ}_{i} ) A [ {a ( {TP}^{ n} )}^{2} + ( 1 - a ) {( {TS}^{ m} )}^{2} ] + K$$

where F(λᵢ) represents a normalized reflection for a wavelength λᵢ with respect to a wavelength λⱼ following bleaching of the visual pigments, A is an absorption factor, a accounts for relative proportion of cones with respect to rods, TP accounts for cone sensitivity, TS accounts for rod sensitivity, n and m are exponents measured respectively from sensitivity curves for scotopic and photopic vision at the given wavelength λᵢ, and K accounts for a contribution from parasitic light. Preferably, values for F(λᵢ) are determined from a known normalized reflection curve, such as the one given in FIG. 3. The number N of unknown variables in such a case would be five: A, a, K, TS, and TP.

When bleaching of the retina is possible, the equation posed in step (d) relating the residual intensity Iᵣ(λᵢ) to the density and relative proportions of the visual pigments is:

$$\frac{{I}_{r} ( {λ}_{i} )}{{I}_{i n} ( {λ}_{i} )} = ( \frac{{I}_{rbleached} ( {λ}_{i} )}{{I}_{i n} ( {λ}_{i} )} - K ) [ {a ( {TP}^{ n} )}^{2} + ( 1 - a ) {( {TS}^{ m} )}^{2} ] + K$$

where I_{rbleached}(λᵢ) is the residual intensity of the residual light beam coming from the retina when in a bleached state, a accounts for relative proportion of cones with respect to rods, TP accounts for cone sensitivity, TS accounts for rod sensitivity, n and m are exponents measured respectively from sensitivity curves for scotopic and photopic vision at the given wavelength λᵢ, and K accounts for a contribution from parasitic light. The number N of unknown variables in the bleaching case would be four: a, K, TS, and TP—the values of I_{rbleached}(λᵢ) being determined through bleaching of the retina in an additional step, before upcoming step (f), described below.
(e) Repeating steps (a) through (d) for a number N of image acquisitions, the illuminating the retina including projecting a light beam of a different wavelength λᵢ and a same incident intensity Iᵢₙ(λᵢ) onto the retina for each acquisition

In both, the case when bleaching is not possible and the case when bleaching is possible, steps (a) through (d) above are repeated to acquire a number N of images. For each iteration, the illuminating the retina of step (a) is done using light of the same incident intensity but of a different wavelength.

The actual repeating may be in part a manual process involving the physical replacement of the light source and recalibration of the incident light intensity or the insertion of a different interferential filter in front of the same light source so as to select a light of a different wavelength. Advantageously, it may involve an automated process controlled by computer means.
(+) Additional step of determining I_{rbleached}(λᵢ) through observation of the retina in a bleached state

In the case when bleaching is possible, as mentioned hereinabove, the method further includes an additional step of determining I_{rbleached}(λᵢ) through observation of the retina in a bleached state.

Preferably, the additional step includes the substeps of:
(i) bleaching the retina;
(ii) illuminating the bleached retina with a light beam of a given incident intensity Iᵢₙ(λᵢ) and a given wavelength λᵢ;
(iii) detecting a residual light beam coming from said bleached retina and acquiring light data from said residual light beam using a photosensing device having a bidimensionnal array of pixels;
(iv) processing said light data acquired by said photosensing device to attribute a residual intensity I_{rbleached}(λᵢ) of said residual light beam to each of said pixels thereby producing a corresponding spatial image of said retina;
(v) repeating steps (i) through (v) for a number N of image acquisitions, said illuminating said retina comprising projecting a light beam of a different wavelength λᵢ and a same incident intensity Iᵢₙ(λᵢ) onto said retina for each acquisition, wherein each of said different wavelengths λᵢ correspond to one of the different wavelengths λᵢ of step (e).

Methods of bleaching the retina are commonly known to those versed in the field. It basically involves illuminating the retina with bright light so as to cause the degeneration of the photopigment rhodopsin resulting in temporary insensitivity to light of the rods while the rhodopsin is regenerated.

In order to determine values for the I_{rbleached}(λᵢ), a second series of N image acquisitions are made following substeps (i) to (v). Substeps (i) to (v) are basically carried out in the same manner as steps (a) to (e) above to obtain this second series of N images which correspond identically to the N images acquired through steps (a) to (e) in practically every aspect but one—the retina in this second series is now in a bleached state.
(f) For each pixel, numerically solving a set of N equations obtained through step (e) for the unknown variables to obtain therefrom the in-vivo spatial measurement of the retina representative of the density and relative proportions of the visual pigments in the retina

Numerically solution of the set of N equations is carried out using a fast, powerful computer. Advantageously, the numerical solution may be carried out by a number of computers, connected in series or preferably in parallel, to optimise calculation time and memory.

According to another aspect of the invention, there is provided a system for in vivo spatial measurement of a retina of an eye of a patient representative of density and relative proportions of visual pigments in the retina.

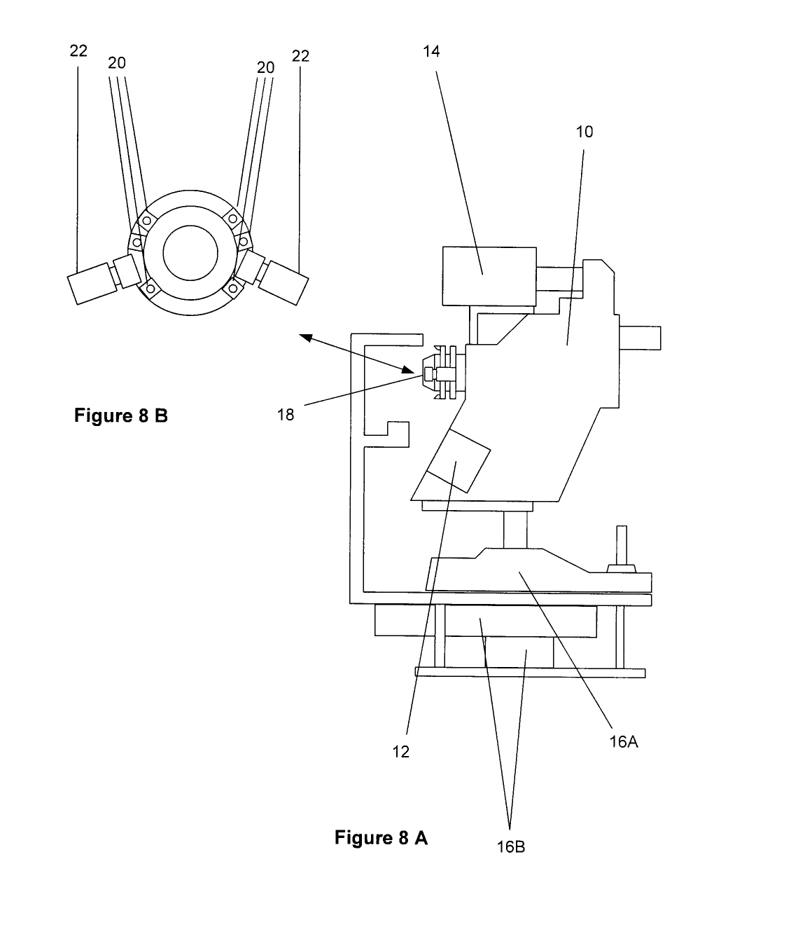

Referring to FIGS. 8A and 8B, the system includes illumination means for illuminating the retina with light of a given wavelength and given incident intensity. The illumination means preferably include a light source. The light source used preferably includes a source of visible light. The source of visible light may be a source of monochromatic visible light, as in the case of a laser. Alternatively, the source may be a source of polychromatic visible light, as in the case of a light source of white light. Interferential filters (12) may be provided for selecting a light of a given wavelength λᵢ. A calibration photometer may also be provided for selecting the incident intensity Iᵢₙ(λᵢ) of the light. Advantageously, the illumination means may be a light source of an ophthalmoscopic camera (10) used to view the eye of the patient.

The present invention also provides a light data acquisition system. The light data acquisition system includes a photosensing device having a bidimensionnal array of pixels for detecting a residual light beam coming from the retina following illumination of the retina and acquiring corresponding light data, a processor for processing light data acquired by each pixel of the photosensing device and attributing a residual intensity Iᵣ(λ) of the residual light beam to each of the pixels thereby producing a corresponding spatial image of the retina, and a controller for controllably producing a number N of spatial images of the retina, each spatial image produced using the illumination means with light of a different given wavelength and same given incident intensity for each image.

In addition to light from the photoreceptor cones and rods found in the retina, the residual light beam may include light from the ocular media and pigment epithelium as well as parasitic light.

Any appropriate photon detector with spatial resolution may embody the photosensing device. Preferably, the photosensing device includes a charge-coupled device (CCD) typically consisting of an integrated circuit containing an array of linked, or coupled, light sensitive pixels which sense light through the photoelectric effect. The integrated circuit records the intensity of light as a variable electric charge. As such, the light data may include electric charge in all its variable detectable forms: voltage, current, etc.

The processor may include an analog-to-digital converter to transform the charges into binary data to be further processed by electronic circuitry such as is found in a computer.

Of course, the photosensing device and processor may be incorporated into a CCD fundus camera (14).

The present invention also provides a data analyser for numerically analysing each pixel of each of the number N of spatial images of the retina. The data analyser is used to pose an equation for each pixel relating the residual intensity Iᵣ(λ) to a number N of unknown variables of interest representative of the density and relative proportions of the visual pigments and to numerically solve for each pixel a set of N equations for the unknown variables to obtain therefrom the in-vivo spatial measurement of the retina representative of the density and relative proportions of the visual pigments in the retina. The data analyser preferably includes a computer and a computer-executable application. Given the complexity of the analysis involved, the computer should be powerful enough to execute a numerical solution of the N equations. Advantageously, the data analyser may include a number of computers connected in series or preferably in parallel to optimise calculation time and memory.

According to an embodiment of the system, the system may include image alignment means for controllably aligning the light source and photosensing device with the eye. The image alignment means include a positioning system for adjustably positioning the light source and the photosensing device along x, y, and z axes. In actuality, the positioning system may be comprised of separate parts: a z-axis translator for vertical translation in the z-axis (16A) and an x-y translation stage for horizontal translation along the x-y axes (16B), as may be the case for aligning the ophthalmoscopic camera (10) (which incorporates the light source) and the associated, connected, CCD fundus camera (14) with the eye. Alternatively, the positioning system may include three independent translators, one for translation along each axis. At least three light-emitting diodes (LEDs) (20) positioned proximate the eye, or specifically the eyepiece (18) of the ophthalmoscopic camera (10) as the case may be, for producing at least three reflections on a cornea of the eye. Two sets of three LEDs may be provided, one set positioned in accordance to a right eye and the other set positioned in accordance to a left eye. The LEDs (20) preferably emit light in the near infrared region of the electromagnetic spectrum so as to not affect the in vivo spatial measurement. A secondary charge-coupled device (CCD) camera (22) for receiving and recording the three reflections is positioned proximate the eye and each set of three LEDs (20). The image alignment means include a position-controller for spatially tracking the three reflections and controlling the positioning system. The position controller may include a computer-executed application and computer. The reflected light from the cornea received from the secondary CCD (22) is processed and analysed by the computer application of the position controller. The image alignment means also include a line-of-sight acquisition system for determining a contour of a pupil of the eye and thereby a line of sight. Here, too, the line-of-sight-acquisition system may include a computer-executed application. Alternatively, it may be accomplished manually by controllably adjusting the relative position of the eye and light source.

### Detailed Description

### Mathematical Analysis

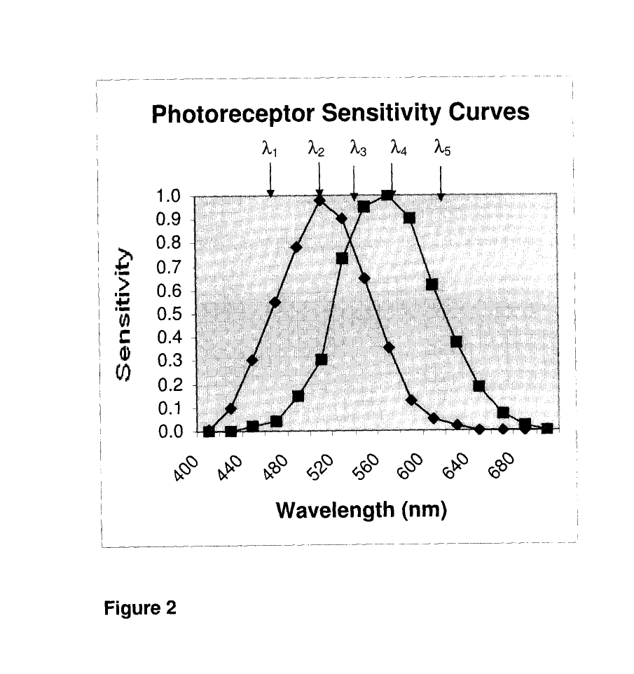

The present invention involves a method and system of sending light of a given incident intensity and wavelength into the eye and treating the residual light coming out of the eye. Given that the aim is to measure from every respect of the retina the proportion of cones and rods, we use sensitivity curves of these two types of photoreceptors to decouple their respective roles during the absorption of light. The three types of cones have different absorption characteristics and must be considered separately. Nonetheless, two simple hypotheses allow the merging of their characteristics in order to arrive at an acceptable solution. On one hand, the blue cones are few in number (≈10%) and are negligible. On the other hand, the characteristics of red cones and green cones being relatively similar (≈50 nm difference), red and green cones are considered in a first approximation as indistinguishable. As a result, the measured value of the absorption of cones is an average value weighted according to their respective spatial density, which is generally in accordance with photopic measurements. FIG. 2 gives the response of these two groups of photoreceptors (i.e., the cones and rods) as a function of the wavelength of light in the visible region of the electromagnetic spectrum (400 nm to 700 nm).

When the light coming out of the eye is absorbed by the cones and the rods, equation (2) is expanded to include the cones and rods. It becomes:

$$\frac{{I}_{r} ( {λ}_{i} )}{{I}_{i n} ( {λ}_{i} )} = A ( {λ}_{i} ) [ {{aT}_{c} ( {λ}_{i} )}^{2} + ( 1 - a ) {{T}_{b} ( {λ}_{i} )}^{2} ] + K \qquad Equation ( 7 )$$

where: a=proportion of cones (varying from 0 to 1)

T_{c}²(λᵢ)=transmission of cones

T_{b}²(λᵢ)=transmission of rods

K=parasitic light

A(λᵢ)=T²ₘₒ(λᵢ)Rₑₚ(λᵢ) (same value as before)

This new equation contains five unknowns (a, A(λᵢ), K, T_{c}, (λᵢ), and T_{b}(λᵢ)), three of which depend on the wavelength. It is possible to express the transmission values of the cones and rods in terms of the wavelength by using the scotopic and photopic sensitivity curves of the human eye. The principle of the method is as previously introduced and the essentials reside in the fact that the following relationships can be established between the transmission and the sensitivity for a given wavelength (λ):

Cones: T_{c}(λᵢ)²=(TPⁿ)²

Rods: T_{b}(λᵢ)²=(TS^{m})²

The exponents n an d m are measured directly from the curves of FIG. 2. Equation (7) can be written as:

$$\frac{{I}_{r} ( {λ}_{i} )}{{I}_{i n} ( {λ}_{i} )} = A ( {λ}_{i} ) [ {a ( {TP}^{ n} )}^{2} + ( 1 - a ) {( {TS}^{ m} )}^{2} ] + K \qquad Equation ( 8 )$$

The variable A (λᵢ) can be evaluated during the bleaching of the visual pigments. Therefore:

$$\frac{{I}_{rbleached} ( {λ}_{i} )}{{I}_{i n} ( {λ}_{i} )} = A ( {λ}_{i} ) + K ,$$

and equation (8) can be written as:

$$\frac{{I}_{r} ( {λ}_{i} )}{{I}_{i n} ( {λ}_{i} )} = ( \frac{{I}_{rbleached} ( {λ}_{i} )}{{I}_{i n} ( {λ}_{i} )} - K ) [ {a ( {TP}^{ n} )}^{2} + ( 1 - a ) {( {TS}^{ m} )}^{2} ] + K \qquad Equation ( 9 )$$

It is worth repeating that this way of proceeding is only valid if the visual pigment can be bleached. (We will consider the case where this is not possible further below.) Pursuant to the preceding mathematical development and considering the number of unknowns (8), two series of four measurements at different wavelengths (λ₁, λ₂, λ₃ and λ₄) must be carried out to determine the variables of interest (A, a, TS, TP and K) so long as for a given wavelength, light of identical intensity is used. At this moment: Iᵢₙ(λᵢ)=Iᵢₙ.

According to the wavelengths (λ₁, λ₂, λ₃, and λ₄) used, we therefore have:

$$\begin{matrix}\frac{{I}_{r} ( {λ}_{1} )}{{I}_{i n}} = A ( {λ}_{1} ) [ {a ( {TP}^{ b} )}^{2} + ( 1 - a ) {( {TS}^{ c} )}^{2} ] + K & Equation ( 10 ) \\ \frac{{I}_{r} ( {λ}_{2} )}{{I}_{i n}} = A ( {λ}_{2} ) [ {a ( {TP}^{ d} )}^{2} + ( 1 - a ) {( {TS}^{ e} )}^{2} ] + K & Equation ( 11 ) \\ \frac{{I}_{r} ( {λ}_{3} )}{{I}_{i n}} = A ( {λ}_{3} ) [ {a ( {TP}^{ f} )}^{2} + ( 1 - a ) {( {TS}^{ g} )}^{2} ] + K & Equation ( 12 ) \\ \frac{{I}_{r} ( {λ}_{4} )}{{I}_{i n}} = A ( {λ}_{4} ) [ {a ( {TP}^{ h} )}^{2} + ( 1 - a ) {( {TS}^{ k} )}^{2} ] + K & Equation ( 13 )\end{matrix}$$

Care was taken to determine the exponents of the transmission coefficients of the cones and rods from the curves of FIG. 2. The unknowns A(λᵢ) can be evaluated by bleaching the pigments of the retina. Measurement of the intensity of the residual light coming from the bleached retina, reduces the preceding equations to the following, given that the exponents are now equal to zero:

$$\begin{matrix}\frac{{I}_{rbleached } ( {λ}_{1} )}{{I}_{i n}} = A ( {λ}_{1} ) + K & Equation ( 14 ) \\ \frac{{I}_{rbleached } ( {λ}_{2} )}{{I}_{i n}} = A ( {λ}_{2} ) + K & Equation ( 15 ) \\ \frac{{I}_{rbleached } ( {λ}_{3} )}{{I}_{i n}} = A ( {λ}_{3} ) + K & Equation ( 16 ) \\ \frac{{I}_{rbleached } ( {λ}_{4} )}{{I}_{i n}} = A ( {λ}_{4} ) + K & Equation ( 17 )\end{matrix}$$

By replacing the values A(λᵢ) in Equations (10) to (13), the final equations used are obtained:

$$\begin{matrix}\frac{{I}_{r} ( {λ}_{1} )}{{I}_{i n}} = ( \frac{{I}_{rbleached} ( {λ}_{1} )}{{I}_{i n}} - K ) [ {a ( {TP}^{ b} )}^{2} + ( 1 - a ) {( {TS}^{ c} )}^{2} ] + K & Equation ( 18 ) \\ \frac{{I}_{r} ( {λ}_{2} )}{{I}_{i n}} = ( \frac{{I}_{rbleached} ( {λ}_{2} )}{{I}_{i n}} - K ) [ {a ( TP )}^{2} + ( 1 - a ) {( {TS}^{ e} )}^{2} ] + K & Equation ( 19 ) \\ \frac{{I}_{r} ( {λ}_{3} )}{{I}_{i n}} = ( \frac{{I}_{rbleached} ( {λ}_{3} )}{{I}_{i n}} - K ) [ {a ( {TP}^{ f} )}^{2} + ( 1 - a ) {( {TS}^{ g} )}^{2} ] + K & Equation ( 20 ) \\ \frac{{I}_{r} ( {λ}_{4} )}{{I}_{i n}} = ( \frac{{I}_{rbleached} ( {λ}_{4} )}{{I}_{i n}} - K ) [ {a ( {TP}^{ h} )}^{2} + ( 1 - a ) {( TS )}^{2} ] + K & Equation ( 21 )\end{matrix}$$

### When Bleaching is not Possible

It is very difficult to bleach the visual pigments of a subject on which one wishes to measure the density of the visual pigments, since the procedure requires a lot of attention and cooperation on the part of the subject. It is illusory to believe that this procedure can be carried out in a routine way in a clinical setting.

The best that can be done to counter this difficulty is either to use normalized reflection curves obtained from the literature or to measure the reflection from the back of the eye at the level of the optical nerve from images of subjects under study (see below). We explain here the procedure to follow by using the results of Delori and Pflibsen (F. C. Delori, and K. P. Pflibsen, “Spectral reflectance of the human ocular fundus”, Applied Optics, 28, 1061-1077, 1989, Table 1, page 1062). FIG. 3 shows the average normalized values, obtained from several subjects, of the reflection at the back of the eye at different wavelengths following bleaching of the visual pigment. It is to be noted that the light used (Iᵢₙ) as well as the parasitic light (K), suitable for different experimental setups, may differ. Under these measurement conditions of the visual pigment at a given wavelength (λᵢ), Equation (9) is rewritten as:

$$\frac{{I}_{r} ( {λ}_{i} )}{{I}_{i n}} = \frac{( \frac{{I}_{rbleached} ( {λ}_{j} )}{{I}_{i n}} - K )}{( \frac{{I}_{rbleached} ( {λ}_{i} )}{{I}_{i n}} - K )} [ {a ( {TP}^{ n} )}^{2} + ( 1 - a ) {( {TS}^{ m} )}^{2} ] + K \qquad Equation ( 22 )$$

where: n and m are measured respectively from the sensitivity curves for scotopic and photopic vision at this given wavelength; and
the quotient

$$\frac{( \frac{{I}_{rbleached} ( {λ}_{j} )}{{I}_{i n}} - K )}{( \frac{{I}_{rbleached} ( {λ}_{i} )}{{I}_{i n}} - K )} = F ( {λ}_{i} ) A$$

represents the normalized reflection
from the back of the eye for the wavelength λᵢ with respect to the wavelength λⱼ where Iᵢₙ(λᵢ)=Iᵢₙ(λⱼ)=Iᵢₙ.

The factors F(λᵢ) can be measured from the curve and the factor A can be determined by adding a new measurement to the above equations (Equations (18) to (22)).

Therefore, the following five equations must be solved:

$$\begin{matrix}\frac{{I}_{r} ( {λ}_{1} )}{{I}_{i n}} = F ( {λ}_{1} ) A [ {a ( {TP}^{ b} )}^{2} + ( 1 - a ) {( {TS}^{ c} )}^{2} ] + K & Equation ( 23 ) \\ \frac{{I}_{r} ( {λ}_{2} )}{{I}_{i n}} = F ( {λ}_{2} ) A [ {a ( TP )}^{2} + ( 1 - a ) {( {TS}^{ d} )}^{2} ] + K & Equation ( 24 ) \\ \frac{{I}_{r} ( {λ}_{3} )}{{I}_{i n}} = F ( {λ}_{3} ) A [ {a ( {TP}^{ e} )}^{2} + ( 1 - a ) {( {TS}^{ f} )}^{2} ] + K & Equation ( 25 ) \\ \frac{{I}_{r} ( {λ}_{4} )}{{I}_{i n}} = F ( {λ}_{4} ) A [ {a ( {TP}^{ g} )}^{2} + ( 1 - a ) {( TS )}^{2} ] + K & Equation ( 26 ) \\ \frac{{I}_{r} ( {λ}_{5} )}{{I}_{i n}} = F ( {λ}_{5} ) A [ {a ( {TP}^{ h} )}^{2} + ( 1 - a ) {( {TS}^{k} )}^{2} ] + K & Equation ( 27 )\end{matrix}$$

### Solution Example

An analytical solution to theses equations is impossible. The steps required for reducing these equations with two unknowns follow.

Reducing five equations down to two allows to define the planes that will intersect at the solution. These operations are repeated for each point of the image. Equations (23) to (27) can be written:

IM(λ₁)=a A TP^{2b}+(1−a)A TS^{2c}+K (Equation 23)

IM(λ₂)=a A TP²+(1−a)A TS^{2d}+K (Equation 24)

IM(λ₃)=a A TP^{2e}+(1−a)A TS^{2f}+K (Equation 25)

IM(λ₄)=a A TP^{2g}+(1−a)A TS²+K (Equation 26)

IM(λ₅)=a A TP^{2h}+(1−a)A TS^{2k}+K (Equation 27)

Taking into account the values from b to k and the values of all of the points of the image (see page 11), the value of K can be extracted from Equation (27):

K=1.436−A((1−a)TP^{1.2}+a TS^{0.04})

This value is substituted into the other equations. New Equation (25) then gives the following value for A:

A=−0.74/(1−a)TP^{1.64}−((1−a)TP^{1.2}+a TS^{0.04})+a TS^{1.64})

Repeating the above procedures with A, the value of a from the new Equation (23) is obtained:

$$a = \frac{( - 3.127 {e}^{16} {TP}^{1 / 5} + 3.643 {e}^{16} Tp 6 / 5 - 5.16 {e}^{15} {TP}^{41 / 25} )}{( \begin{matrix}- 3.127 \times 31.909 {e}^{16} {TP}^{1 / 5} + 3.642 \times 31.919 {e}^{16} {TP}^{6 / 5} - \\ 5.16 \times 31.909 {e}^{15} {TP}^{41 / 25} - 3.643 \times 31.909 {e}^{16} {TS}^{1 / 25} + \\ 3.127 \times 31.909 {e}^{16} {TS}^{6 / 5} + 5.16 \times 31.909 {e}^{15} {TS}^{41 / 25}\end{matrix} )}$$

Substituting this value into equations (24) and (26) yields the values of IM(λ₂and IM(λ₄):

$$IM ( {λ}_{2} ) = \frac{( \begin{matrix}{TP}^{0.6} ( 5.222 {TS}^{1 / 25} - 4.482 {TS}^{6 / 5} - 0.74 {TS}^{41 / 25} ) + \\ {TP}^{1.2} ( 4.919 {TS}^{1 / 25} - 4.222 {TS}^{6 / 5} - 0.697 {TS}^{41 / 25} ) + \\ {TP}^{1.64} ( - 10.141 {TS}^{1 / 25} + 8.704 {TS}^{6 / 5} + 1.436 {TS}^{41 / 25} ) + \\ {TP}^{6 / 5} ( - 4.919 {TS}^{0.04} + 10.141 {TS}^{1.64} - 5.222 {TS}^{2} ) + \\ {TP}^{41 / 25} ( 0.697 {TS}^{0.04} - 1.436 {TS}^{1.64} + 0.740 {TS}^{2} ) + \\ {TP}^{1 / 5} ( 4.222 {TS}^{0.04} - 8.704 {TS}^{1.64} + 4.482 {TS}^{2} )\end{matrix} )}{( \begin{matrix}{TP}^{1.2} ( 7.06 {TS}^{1 / 25} - 6.06 {TS}^{6 / 5} - {TS}^{41 / 25} ) + \\ {TP}^{1.64} ( - 7.06 {TS}^{1 / 25} + 6.06 {TS}^{6 / 5} + {TS}^{41 / 25} ) + \\ {TP}^{1 / 5} ( 6.06 {TS}^{0.04} - 6.06 {TS}^{1.64} ) + \\ {TP}^{41 / 25} ( {TS}^{0.04} - {TS}^{1.64} ) + \\ {TP}^{6 / 5} ( - 7.06 {TS}^{0.04} + 7.06 {TS}^{1.64} )\end{matrix} )}$$

$$IM ( {λ}_{4} ) = \frac{( \begin{matrix}{TP}^{2} ( 5.222 {TS}^{1 / 25} - 4.482 {TS}^{6 / 5} - 0.74 {TS}^{41 / 25} ) + \\ {TP}^{1.2} ( 4.919 {TS}^{1 / 25} - 4.222 {TS}^{6 / 5} - 0.697 {TS}^{41 / 25} ) + \\ {TP}^{1.64} ( - 10.141 {TS}^{1 / 25} + 8.704 {TS}^{6 / 5} + 1.436 {TS}^{41 / 25} ) + \\ {TP}^{1 / 5} ( 4.222 {TS}^{0.04} + 4.482 TS - 8.704 {TS}^{1.64} ) + \\ {TP}^{41 / 25} ( 0.697 {TS}^{0.04} + 0.740 TS - 1.436 {TS}^{1.64} ) + \\ {TP}^{6 / 5} ( - 4.919 {TS}^{0.04} - 5.222 TS + 10.141 {TS}^{1.64} )\end{matrix} )}{( \begin{matrix}{TP}^{1.2} ( 7.06 {TS}^{1 / 25} - 6.06 {TS}^{6 / 5} - {TS}^{41 / 25} ) + \\ {TP}^{1.64} ( - 7.06 {TS}^{1 / 25} + 6.06 {TS}^{6 / 5} + {TS}^{41 / 25} ) + \\ {TP}^{1 / 5} ( 6.06 {TS}^{0.04} - 6.06 {TS}^{1.64} ) + \\ {TP}^{41 / 25} ( {TS}^{0.04} - {TS}^{1.64} ) + \\ {TP}^{6 / 5} ( - 7.06 {TS}^{0.04} + 7.06 {TS}^{1.64} )\end{matrix} )}$$

it becomes a matter of solving the equations numerically. A precise example of a simulation (without noise) for a single point of the image is given here.

For illustration purposes, we have chosen the following constants:
b=0.6 c=0.1 d=0.3 e=0.82 f=0.82 g=0.5 h=0.02 k=0.6
F(λ₁)=0.48 F(λ₂)=0.68 F(λ₃)=0.90 (λ₄)=1.07 F(λ₅)=2.20

The values of the variables this particular point of the retina are:
A=2 a=0.4 K=0.5 TS=0.3 TP=0.2

Under these conditions, Equations (23) to (27) yield the corresponding values of this point:

$$IM ( {λ}_{1} ) = \frac{{I}_{r} ( {λ}_{1} )}{{I}_{i n} F ( {λ}_{1} )} = 1.008$$

$$IM ( {λ}_{2} ) = \frac{{I}_{r} ( {λ}_{2} )}{{I}_{i n} F ( {λ}_{2} )} = 0.860$$

$$IM ( {λ}_{3} ) = \frac{{I}_{r} ( {λ}_{3} )}{{I}_{i n} F ( {λ}_{3} )} = 0.677$$

$$IM ( {λ}_{4} ) = \frac{{I}_{r} ( {λ}_{4} )}{{I}_{i n} F ( {λ}_{4} )} = 0.808$$

$$IM ( {λ}_{5} ) = \frac{{I}_{r} ( {λ}_{5} )}{{I}_{i n} F ( {λ}_{5} )} = 2.560$$

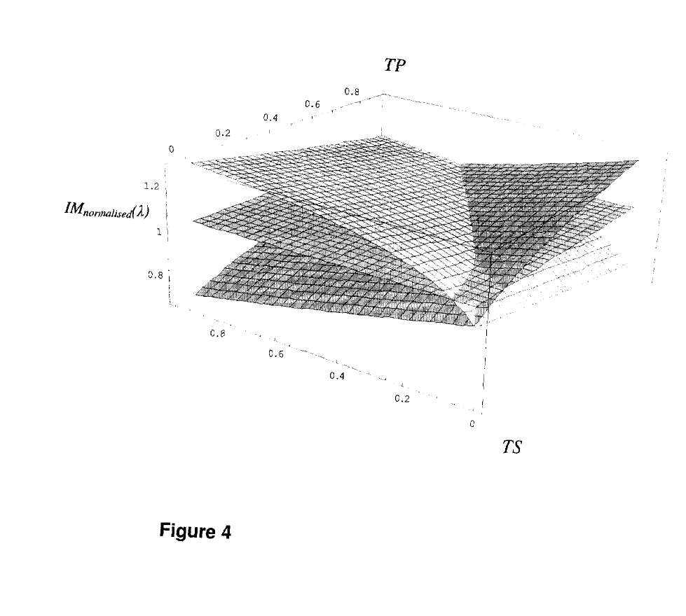

During a densitometry measurement, these preceding values are given by measurement devices and it is simply a matter of proceeding in reverse to find the corresponding values: A, a, K, TS and TP. It was shown earlier in the Solution Example section that it is possible to isolate the factors A, a and K in order to be able to express the two variables TS and TP as a function of the values: IM(λ₁), IM(λ₂), IM(λ₃), IM(λ₄), and IM(λ₅). The two resulting equations are very complex, but knowing that the values of TS and TP are somewhere in the range from 0 to 1, it is sufficient to calculate the values predicted by the two equations for all the possible values of TS and TP. The intersection point of the two planes calculated thusly in a required horizontal plane, yield the desired solution. FIG. 4 shows the result of our simulation. The intersection point is located at TS=0.3 and TP=0.2, as required.

### Real Measurements

While taking real measurements, the fact that the absorption factor A in the equations is somewhat dependent on the wavelength should be taken into account. Two solutions for finding the correction factors are outlined.

The best way of proceeding consists of bleaching the visual pigments of each subject at the start of the experiment and taking four images using light of the required wavelength. Once this is done, the pigments are transparent and equations (10) to (13) are used for computing the required parameters (A, a, TP, TS and K).

The second solution consists of correcting the values of A using the normalised reflection values from the back of the eye obtained from the literature. FIG. 3 gives the normalised values of the reflections from the back of the eye obtained by Delori and Pflibsen 1989 (F. C. Delori, and K. P. Pflibsen, “Spectral reflectance of the human ocular fundus”, Applied Optics, 28, 1061-1077, 1989, Table 1, page 1062). It should be noted that these values were obtained from subjects having undergone bleaching of the visual pigment.

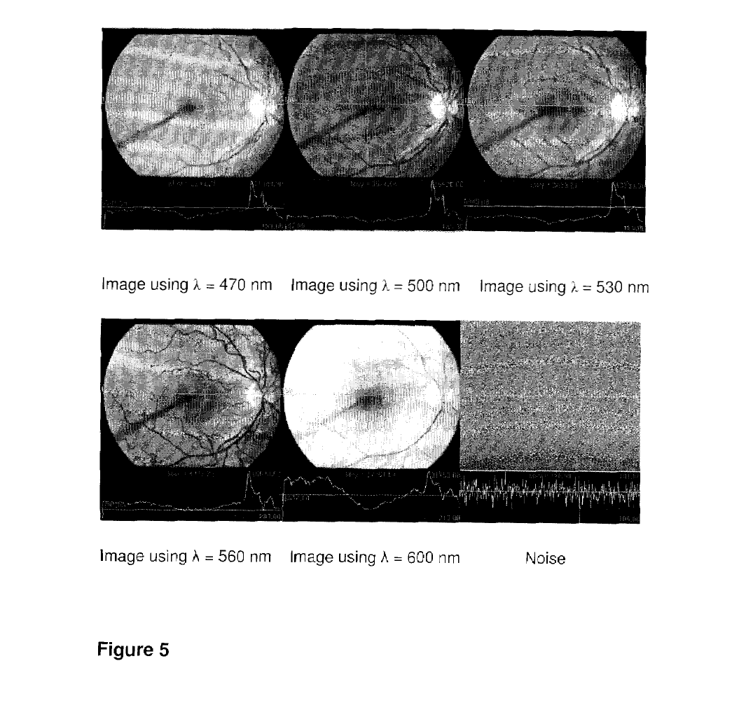

The results in FIG. 5 were obtained from a normal subject and they show the initial five images (in addition to the background noise image) and the profile information of line 150 of each image. Correction factors were applied to the images taking into account the optics used, the non-linearities of the CCD and the calibration photometer used to select the desired light intensities. The details of the latter are not given here explicitly since they are commonly known in the optics domain.

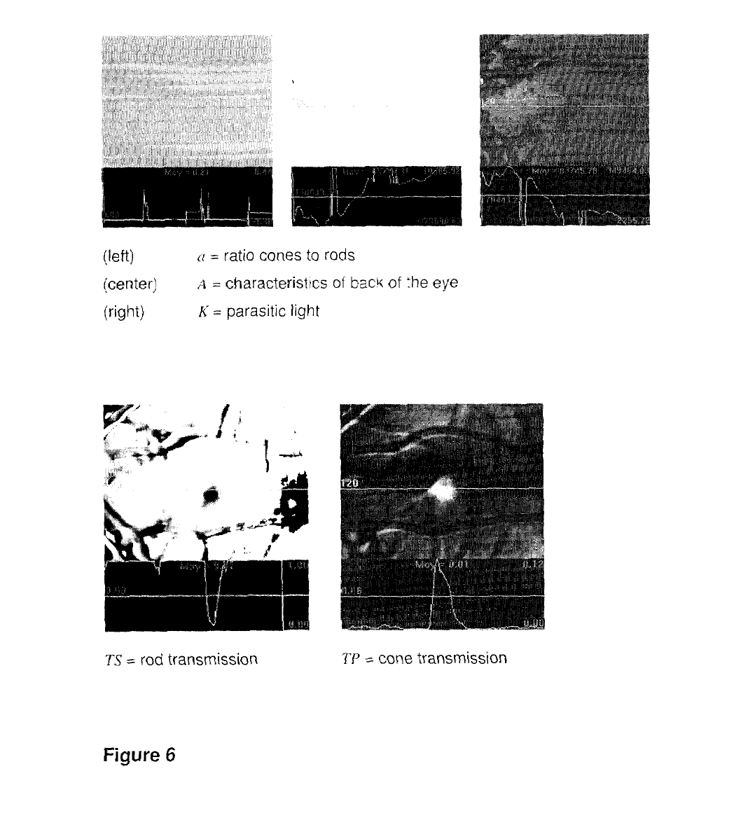

At the time of this experiment, the CCD images of the retina were sufficiently well aligned so that we cannot detect differences in position from image to image of the fine details of the blood vessels and the optical nerve (white disc at the center right). This result was obtained thanks to the tuning of an eye tracking system described further below. The method of analysis being differential, reflections and structural defects do not distort the true values of the pigment density. This was demonstrated through stimulation measurements of the human retinas. The purpose of the results presented here is to demonstrate the feasibility of the technique and to illustrate the preliminary results obtained. Examples of obtained results for the parameters: a, A, K, TS and TP as well as the intensity profiles of each image result for line 120 are given in FIG. 6.

### Positioning of the Eye

In order to assure that the different images are well aligned at the time of taking of the images, the following three controls were carried out:

1. Control of the back-of-the-eye camera
Instead of asking the subject to move in order to better align the images on the CCD camera (14) (also referred to as the CCD fundus camera), the associated ophthalmoscopic camera (10) to which the CCD fundus camera (14) is connected is adjusted along the X, Y, and Z axes with the aid of translation tables (16B and 16A), as shown in FIGS. 8A and 8B.

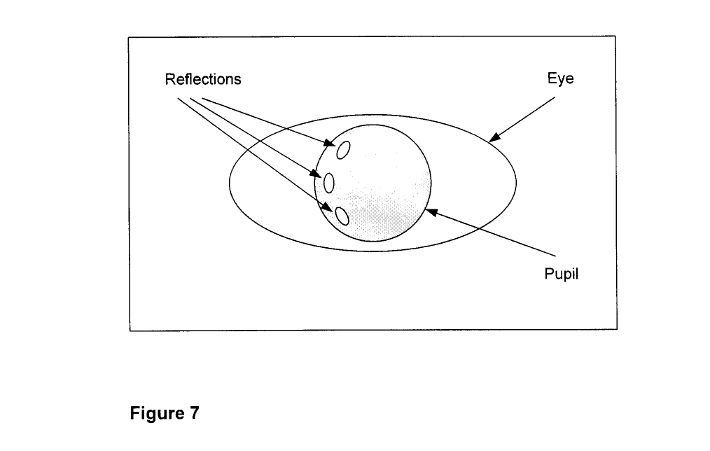

2. Control of the pupil position
Software permitting to position the pupil so as to always be viewed in the same manner by the ophthalmoscopic camera (10) and associated CCD fundus camera (14) was developed. This was done by positioning three infrared LEDs (900-nm light emitting diodes) near the ophthalmoscopic camera (10) in such a way that they produce reflections on the cornea that are captured by a secondary CCD camera (22) sensitive to infrared and positioned at the edge of the ophthalmoscopic camera (10). The translation tables (16A and 16B) are controlled by tracking the position of these points using appropriate trigonometric calculations. FIG. 7 shows the three reflections (small ellipses) on the pupil.

3. Control of the line of sight
Software was developed which determines the contour of the pupil simply by locating its center. This information allows one to find the line of sight and to ascertain that it is the same for all of the pictures of the back of the eye.

During the acquisition of the first image, the locations of the light reflections and the line of sight are stored in memory so that each subsequent image will have the same trigonometric parameters as the first.

### Alternate Embodiments

The method explained here can be generalized and used to find the proportion of the rods and the three types of cones at any point within the eye. This would require taking nine images (given that there would be nine unknowns) and a subsequent enormous calculation time. The method can also be used for measuring the density of either only the cones (TP) or only the rods (TS). In this case, it is a relatively simple matter of solving three equations for three unknowns

Moreover, the method can be used to measure the proportion of red cones and green cones in the fovea since this region is deprived of rods and blue cones. In this case, it is a matter of using the absorption curves of these cones rather than the photopic and scotopic characteristics given on page 6 providing appropriate wavelengths are selected when taking the pictures.

From a clinical point of view, the values of A (characteristic of the back of the eye) and K (parasitic light) can be as useful as the a, TS, and TP values since they can serve as a means of comparing the characteristics of the back of the eye and the dispersion of light by the eye of an individual to that of another individual member of a large group according to the particular pathology.

The “lighting” of the eye could be carried out using either white light or a combination of coloured lights (preferably by the sweeping of several lasers) and interferential filters can be used to select the required images for analysis. This method would eliminate the problems of alignment, but would necessitate a more costly apparatus.

It would seem that lighting (or sweeping) by laser (J. Fortin, “Évaluation non effractive des pigments visuels au moyen d'un densimètre à images video”, PhD Thesis, Laval University (Canada), 1992) would either greatly reduce the parasitic light (K) or render it negligible. If this were the case, the measurement method of the residual variables (A, TS, TP, and a) would require one less wavelength measurement and as such only six measurements need to be taken during bleaching and only four if normalized values are used.

Numerous modifications could be made to any of the embodiments described hereinabove without departing from the scope of the present invention as defined in the appended claims.

1. A method for obtaining an in-vivo spatial measurement of a retina of an eye of a patient representative of density and relative proportions of visual pigments in said retina, the method comprising the steps of:
(a) illuminating said retina with a light beam of a given incident intensity Iᵢₙ(λᵢ) and a given wavelength λᵢ;
(b) detecting a residual light beam coming from said retina and acquiring light data from said residual light beam using a photosensing device having a bidimensionnal array of pixels;
(c) processing said light data acquired by said photosensing device to attribute a residual intensity Iᵣ(λᵢ) of said residual light beam to each of said pixels, thereby producing a corresponding spatial image of said retina;
(d) for each pixel, posing an equation relating the residual intensity Iᵣ(λᵢ) to a number N of unknown variables of interest representative of said density and relative proportions of the visual pigments;
(e) repeating steps (a) through (d) for a number N of image acquisitions, said illuminating said retina comprising projecting a light beam of a different wavelength λᵢ and a same incident intensity Iᵢₙ(λᵢ) onto said retina for each acquisition; and
(f) for each pixel, numerically solving a set of N equations obtained through step (e) for the unknown variables to obtain therefrom the in-vivo spatial measurement of the retina representative of the density and relative proportions of said visual pigments in said retina.

2. The method according to claim 1, wherein the processing of step (c) comprises correcting said spatial images for non-linearities of the photosensing device.

3. The method according to claim 1, wherein said equation posed in step (d) relating the residual intensity Iᵣ(λᵢ) to said density and relative proportions of the visual pigments is:
$$\frac{{I}_{r} ( {λ}_{i} )}{{I}_{i n} ( {λ}_{i} )} = F ( {λ}_{i} ) A [ {a ( {TP}^{ n} )}^{2} + ( 1 - a ) {( {TS}^{ m} )}^{2} ] + K$$
where F(λᵢ) represents a normalized reflection for a wavelength λᵢ with respect to a wavelength λⱼ following bleaching of the visual pigments, A is an absorption factor, a accounts for relative proportion of cones with respect to rods, TP accounts for cone sensitivity, TS accounts for rod sensitivity, n and m are exponents measured respectively from sensitivity curves for scotopic and photopic vision at the given wavelength λᵢ, and K accounts for a contribution from parasitic light.

4. The method according to claim 3, wherein values for F(λᵢ) are determined from a known normalized reflection curve.

5. The method according to claim 3, wherein said number N of unknown variables is five and said unknown variables are A, a, K, TS, and TP.

6. The method according to claim 3, wherein the numerically solving the N equations of step (f) comprises correcting for the wavelength dependence of A.

7. The method according to claim 1, wherein said equation posed in step (d) relating the residual intensity Iᵣ(λᵢ) to said density and relative proportions of the visual pigments is:
$$\frac{{I}_{r} ( {λ}_{i} )}{{I}_{i n} ( {λ}_{i} )} = ( \frac{{I}_{rbleached} ( {λ}_{i} )}{{I}_{i n} ( {λ}_{i} )} - K ) [ {a ( {TP}^{ n} )}^{2} + ( 1 - a ) {( {TS}^{ m} )}^{2} ] + K$$
where I_{rbleached}(λᵢ) is the residual intensity of the residual light beam coming from the retina when in a bleached state, a accounts for relative proportion of cones with respect to rods, TP accounts for cone sensitivity, TS accounts for rod sensitivity, n and m are exponents measured respectively from sensitivity curves for scotopic and photopic vision at the given wavelength λᵢ, and K accounts for a contribution from parasitic light.

8. The method according to claim 7, further comprising an additional step before step (f) of determining I_{rbleached}(λᵢ) through observation of the retina in a bleached state.

9. The method according to claim 8, wherein said additional step comprises the substeps of:
(i) bleaching the retina;
(ii) illuminating said bleached retina with a light beam of a given incident intensity Iᵢₙ(λᵢ) and a given wavelength λᵢ;
(iii) detecting a residual light beam coming from said bleached retina and acquiring light data from said residual light beam using a photosensing device having a bidimensionnal array of pixels;
(iv) processing said light data acquired by said photosensing device to attribute a residual intensity I_{rbleached}(λᵢ) of said residual light beam to each of said pixels thereby producing a corresponding spatial image of said retina;
(v) repeating steps (i) through (v) for a number N of image acquisitions, said illuminating said retina comprising projecting a light beam of a different wavelength λᵢ and a same incident intensity Iᵢₙ(λᵢ) onto said retina for each acquisition, wherein each of said different wavelengths λᵢ corresponds to one of the different wavelengths λᵢ of step (e).

10. The method according to claim 9, wherein said number N of unknown variables is four and said unknown variables are a, K, TS, and TP.

11. A system for in vivo spatial measurement of a retina of an eye of a patient representative of density and relative proportions of visual pigments in said retina, said system comprising:
illumination means for illuminating said retina with light of a given incident intensity Iᵢₙ(λ) and a given wavelength A;
a light data acquisition system comprising:
a photosensing device for detecting a residual light beam coming from said retina and acquiring corresponding light data, said photosensing device having a bidimensionnal array of pixels;
a processor for processing light data acquired by each pixel of said photosensing device and attributing a residual intensity Iᵣ(λ) of said residual light beam to each of said pixels thereby producing a corresponding spatial image of said retina; and
a controller for controllably producing a number N of spatial images of the retina, each spatial image produced using said illumination means with light of a different given wavelength and same given incident intensity for each image; and
a data analyser for numerically analysing each pixel of each of said number N of spatial images of the retina, said data analyser posing an equation for each pixel relating the residual intensity Iᵣ(λ) to a number N of unknown variables of interest representative of said density and relative proportions of the visual pigments and numerically solving for each pixel a set of N equations for the unknown variables to obtain therefrom the in-vivo spatial measurement of the retina representative of the density and relative proportions of said visual pigments in said retina.

12. A system according to claim 11, wherein said illumination means comprises a light source.

13. A system according to claim 12, wherein said illumination means further comprises at least one interferential filter for selecting said light of a given wavelength.

14. A system according to claim 13, wherein said light source comprises a source of visible light.

15. A system according to claim 13, wherein said light source comprises a source of white light.

16. A system according to claim 13, wherein said light source comprises a source of polychromatic light.

17. A system according to claim 12, wherein said light source comprises a source of monochromatic light.

18. A system according to claim 12, wherein said light source comprises a laser.

19. A system according to claim 12, wherein said illumination means comprises a calibration photometer for selecting said given incident intensity.

20. A system according to claim 11, comprising an ophthalmoscopic camera, said ophthalmoscopic camera incorporating said illuminations means.

21. A system according to claim 20, comprising a charge-coupled device (CCD) fundus camera associated with said ophthalmoscopic camera, said CCD fundus camera incorporating said photosensing device and said processor.

22. A system according to claim 21, further comprising image alignment means for controllably aligning said ophthalmoscopic camera with said eye, said image alignment means comprising:
a positioning system for adjustably positioning the ophthalmoscopic camera along x, y, and z axes;
at least three infrared light-emitting diodes (LEDs) for producing at least three reflections on a cornea of the eye, said at least three LEDs being positioned proximate an eyepiece of the ophthalmoscopic camera;
a secondary charge-coupled device (CCD) camera for receiving and recording said at least three reflections, said secondary CCD camera being associated with the at least three LEDs and positioned proximate the eyepiece of the ophthalmoscopic camera;
a position-controller for spatially tracking said at least three reflections and controlling said positioning system; and
a line-of-sight acquisition system for determining a contour of a pupil of the eye and thereby a line of sight.

23. A system according to claim 22, wherein said data analyser comprises computer means.

24. A system according to claim 11, comprising a charge-coupled device (CCD) fundus camera, said CCD fundus camera incorporating said photosensing device and said processor.

25. A system according to claim 11, further comprising image alignment means for controllably aligning said illumination means and said photosensing device with said eye, said image alignment means comprising:
a positioning system for adjustably positioning the illumination means and the photosensing device along x, y, and z axes;
at least three light-emitting diodes (LEDs) for producing at least three reflections on a cornea of the eye, said at least three LEDs being positioned proximate the eye;
a secondary charge-coupled device (CCD) camera for receiving and recording said at least three reflections, said secondary CCD camera being associated with the at least three LEDs and positioned proximate the eye;
a position-controller for spatially tracking said at least three reflections and controlling said positioning system; and
a line-of-sight acquisition system for determining a contour of a pupil of the eye and thereby a line of sight.

26. A system according to claim 11, wherein said data analyser comprises computer means.
